(19) United States
(12) Patent Application Publication
(10) Pub. No.: US 2017/0304819 A1
(43) Pub. Date: Oct. 26, 2017

(54) CONTAINER ASSEMBLY AND CONTAINER ASSEMBLY KIT

(71) Applicant: SEIKO EPSON CORPORATION, Tokyo (JP)

(72) Inventors: Toshiro MURAYAMA, Fujimi-machi (JP); Fumio TAKAGI, Chino-shi (JP)

(21) Appl. No.: 15/513,078

(22) PCT Filed: Sep. 30, 2015
(86) PCT No.: PCT/JP2015/004974

§ 371 Date: Mar. 21, 2017

(30) Foreign Application Priority Data
Sep. 30, 2014 (JP) 2014-199561

(51) Int. Cl. B01L 3/00; C12Q 1/68

(52) U.S. Cl. CPC B01L 3/502; C12Q 1/686; C12Q 1/6848; B01L 2300/0848; B01L 2200/16; B01L 2200/141; B01L 2200/0689; B01L 2400/043; B01L 2300/087

(57) ABSTRACT

A container assembly prevents a situation in which an aqueous liquid layer is contaminated by a component of another aqueous liquid layer even when stored for a long time. A container assembly has a structure in which an adsorption container, a reaction container, a washing container, and an elution container are joined to form a flow channel through which the target nucleic acid is moved. The washing container may include a first washing container, a second washing container, and a third washing container. The washing containerseal-tightly holds a first washing liquid, a second washing liquid, a third washing liquid, a first oil, a second oil, a third oil, and a fourth oil, the first oil, the second oil, the third oil, and the fourth oil being immiscible with the first washing liquid, the second washing liquid, and the third washing liquid. The first washing liquid, the second washing liquid, and the third washing liquid are liquids with which a magnetic bead on which a nucleic acid is adsorbed is washed. The elution container seal-tightly holds an eluent and a fluid that is immiscible with the eluent. The eluent is a liquid with which the target nucleic acid is eluted from the magnetic bead.

### CROSS REFERENCE TO RELATED APPLICATIONS

This application is a U.S. National Phase Application under 35 U.S.C. 371 of International Application No. PCT/JP2015/004974 filed on Sep. 30, 2015 and published in English as WO 2016/051793 A1 on Apr. 7, 2016. This application claims priority to Japanese Patent Application No. 2014-199561 filed on Sep. 30, 2014. The entire disclosures of all of the above applications are incorporated herein by reference.

### TECHNICAL FIELD

The present invention relates to a container assembly that has a flow channel through which a biological substance is moved, and a container assembly kit that is used to assemble the container assembly.

### BACKGROUND ART

Polymerase chain reaction (PCR) technology has been established in the field of biochemistry. In recent years, PCR amplification accuracy and PCR detection sensitivity have been improved, and it has become possible to amplify, detect, and analyze a trace amount of a sample (e.g., DNA). The PCR technology subjects a solution (reaction solution) that includes the amplification target nucleic acid (target nucleic acid) and a reagent to thermal cycling to amplify the target nucleic acid. The solution is normally subjected to PCR thermal cycling at two or three different temperatures.

At present, the presence or absence of infection (e.g., influenza) is normally determined using a rapid test kit (e.g., immunochromatography). However, since the determination accuracy may be insufficient when such a rapid test kit is used, it has been desired to use PCR technology that can achieve higher examination accuracy when determining the presence or absence of infection.

In recent years, a device in which aqueous liquid layers and water-insoluble gel layers are alternately stacked within a capillary has been proposed as a device used for PCR technology and the like (see WO2012/086243). In this case, a magnetic material particle to which a nucleic acid adheres is passed through the capillary to purify the nucleic acid. However, such a device has a problem in that a component of one aqueous liquid layer may gradually diffuse through the gel layer, and contaminate another aqueous liquid layer when stored for a long time.

### SUMMARY OF INVENTION

### Technical Problem

An object of the invention is to provide a container assembly that prevents a situation in which an aqueous liquid layer is contaminated by a component of another aqueous liquid layer even when stored for a long time.

### Solution to Problem

The invention was conceived in order to solve at least some of the above problems, and may be implemented as described below (see the following aspects and application examples).

### Application Example 1

According to one aspect of the invention, a container assembly includes:

a flow channel through which a biological substance is moved, the flow channel being formed by joining a washing container that seal-tightly holds a washing liquid and a fluid that is immiscible with the washing liquid, and an elution container that seal-tightly holds an eluent and a fluid that is immiscible with the eluent, the washing liquid being a liquid with which a substance-binding solid-phase carrier on which the biological substance is adsorbed is washed, and the eluent being a liquid with which the biological substance is eluted from the substance-binding solid-phase carrier.

According to the container assembly, since the washing container and the elution container respectively seal-tightly hold the contents until the washing container and the elution container are joined, it is possible to prevent a situation in which the eluent is contaminated by the washing liquid. Since the washing liquid and the eluent are not mixed with each other due to the presence of the fluid that is immiscible with the washing liquid and the eluent even after the washing container and the elution container have been joined, it is possible to prevent a situation in which the eluent is contaminated by the washing liquid on condition that the container assembly is used immediately after assembly.

### Application Example 2

In the container assembly, the washing container may include two or more separate washing containers that respectively seal-tightly hold the washing liquid and the fluid that is immiscible with the washing liquid, a separate washing container among the two or more separate washing containers may be joined to the elution container, and another separate washing container among the two or more separate washing containers may be joined to the separate washing container that is joined to the elution container.

According to the container assembly, since the substance-binding solid-phase carrier can be washed two or more times using the washing liquids that are respectively held by two or more separate washing containers, it is possible to improve the washing efficiency. It is also possible to prevent a situation in which each washing liquid is contaminated until the separate washing containers are joined. Since the washing liquids are not mixed with each other due to the presence of the fluid that is immiscible with the washing liquids even after the separate washing containers have been joined, it is possible to prevent a situation in which each washing liquid is contaminated on condition that the container assembly is used immediately after assembly.

### Application Example 3

The container assembly may further include an adsorption container that seal-tightly holds an adsorbent and a fluid that is immiscible with the adsorbent, the adsorption container being joined to the washing container to form the flow channel, the adsorbent may be a liquid in which a nucleic acid is adsorbed on the substance-binding solid-phase carrier, and the adsorption container may be joined to an end of the washing container that differs from an end to which the elution container is joined.

According to the container assembly, since the adsorption container seal-tightly holds the contents until the adsorption container and the washing container are joined, it is possible to prevent a situation in which the washing liquid is contaminated by the adsorbent. Since the adsorbent and the washing liquid are not mixed with each other due to the presence of the fluid that is immiscible with the adsorbent and the washing liquid even after the adsorption container and the washing container have been joined, it is possible to prevent a situation in which the washing liquid is contaminated by the adsorbent on condition that the container assembly is used immediately after assembly.

### Application Example 4

The container assembly may further include a reaction container that seal-tightly holds an oil that is immiscible with the eluent, the reaction container being joined to the elution container to form the flow channel, and the reaction container may be joined to an end of the elution container that differs from an end to which the washing container is joined.

According to the container assembly, washing, elution, and a reaction can be sequentially effected by joining the reaction container to the elution container. Since the reaction container and the elution container are provided separately, it is possible to reduce the length of the elution container.

### Application Example 5

In the container assembly, one end of a part of the washing container that forms the flow channel may be inserted into one end of a part of the elution container that forms the flow channel, and part of an outer wall of the one end of the part of the washing container that forms the flow channel may come in contact with part of an inner wall of the one end of the part of the elution container that forms the flow channel.

According to the container assembly, since the outer wall of one end of the part of the washing container that forms the flow channel comes in contact with the inner wall of one end of the part of the elution container that forms the flow channel, it is possible to easily move the substance-binding solid-phase carrier from the washing container to the elution container.

### Application Example 6

According to another aspect of the invention, a container assembly includes:

a flow channel through which a biological substance is moved, the flow channel being formed by joining an adsorption container that seal-tightly holds an adsorbent and a fluid that is immiscible with the adsorbent, and a washing container that seal-tightly holds a washing liquid and a fluid that is immiscible with the washing liquid, the adsorbent being a liquid in which a nucleic acid is adsorbed on a substance-binding solid-phase carrier, and the washing liquid being a liquid with which the substance-binding solid-phase carrier on which the biological substance is adsorbed is washed.

According to the container assembly, since the adsorption container and the washing container respectively seal-tightly hold the contents until the adsorption container and the washing container are joined, it is possible to prevent a situation in which the washing liquid is contaminated by the adsorbent. Since the washing liquid and the adsorbent are not mixed with each other due to the presence of the fluid that is immiscible with the washing liquid and the adsorbent even after the adsorption container and the washing container have been joined, it is possible to prevent a situation in which the washing liquid is contaminated by the adsorbent on condition that the container assembly is used immediately after assembly.

### Application Example 7

According to another aspect of the invention, a container assembly includes: a flow channel through which a biological substance is moved, the flow channel being formed by joining an adsorption container that seal-tightly holds an adsorbent and a fluid that is immiscible with the adsorbent, and an elution container that seal-tightly holds an eluent and a fluid that is immiscible with the eluent, the adsorbent being a liquid in which a nucleic acid is adsorbed on a substance-binding solid-phase carrier, and the eluent being a liquid with which the biological substance is eluted from the substance-binding solid-phase carrier.

According to the container assembly, since the adsorption container and the elution container respectively seal-tightly hold the contents until the adsorption container and the elution container are joined, it is possible to prevent a situation in which the eluent is contaminated by the adsorbent. Since the eluent and the adsorbent are not mixed with each other due to the presence of the fluid that is immiscible with the eluent and the adsorbent even after the adsorption container and the elution container have been joined, it is possible to prevent a situation in which the eluent is contaminated by the adsorbent on condition that the container assembly is used immediately after assembly.

### Application Example 8

According to another aspect of the invention, a container assembly includes:

a flow channel through which a biological substance is moved, the flow channel being formed by joining a first washing container that seal-tightly holds a first washing liquid and a fluid that is immiscible with the first washing liquid, and a second washing container that seal-tightly holds a second washing liquid and a fluid that is immiscible with the second washing liquid, the second washing liquid differing from the first washing liquid,

the first washing liquid and the second washing liquid being a liquid with which a substance-binding solid-phase carrier on which the biological substance is adsorbed is washed.

According to the container assembly, since the first washing container and the second washing container respectively seal-tightly hold the contents until the first washing container and the second washing container are joined, it is possible to prevent a situation in which the second washing liquid is contaminated by the first washing liquid. Since the first washing liquid and the second washing liquid are not mixed with each other due to the presence of the fluid that is immiscible with the first washing liquid and the second washing liquid even after the first washing container and the second washing container have been joined, it is possible to prevent a situation in which the second washing liquid is contaminated by the first washing liquid on condition that the container assembly is used immediately after assembly.

### Application Example 9

According to another aspect of the invention, a container assembly kit includes:

a washing container that seal-tightly holds a washing liquid and a fluid that is immiscible with the washing liquid; and

an elution container that seal-tightly holds an eluent and a fluid that is immiscible with the eluent,

the washing liquid being a liquid with which a substance-binding solid-phase carrier on which the biological substance is adsorbed is washed,

the eluent being a liquid with which the biological substance is eluted from the substance-binding solid-phase carrier, and

the washing container and the elution container being able to be joined to form a flow channel through which the biological substance is moved.

According to the container assembly kit, since the washing container and the elution container respectively seal-tightly hold the contents, it is possible to prevent a situation in which the eluent is contaminated by the washing liquid until the washing container and the elution container are joined.

### Application Example 10

In the container assembly kit, the washing container may include two or more separate washing containers that respectively seal-tightly hold the washing liquid and the fluid that is immiscible with the washing liquid, a separate washing container among the two or more separate washing containers may be able to be joined to the elution container, and another separate washing container among the two or more separate washing containers may be able to be joined to the separate washing container that is joined to the elution container.

According to the container assembly kit, it is possible to prevent a situation in which each washing liquid is contaminated until the separate washing containers are joined. It is also possible to adjust the washing accuracy corresponding to the user's demand since two or more separate washing containers are used.

### Application Example 11

The container assembly kit may further include an adsorption container that seal-tightly holds an adsorbent and a fluid that is immiscible with the adsorbent, the adsorbent may be a liquid in which a nucleic acid is adsorbed on the substance-binding solid-phase carrier, and the adsorption container may be able to be joined to an end of the washing container that differs from an end to which the elution container is joined.

According to the container assembly kit, since the adsorption container seal-tightly holds the contents, it is possible to prevent a situation in which the washing liquid is contaminated by the adsorbent until the adsorption container and the washing container are joined.

### Application Example 12

The container assembly kit may further include a reaction container that seal-tightly holds an oil that is immiscible with the eluent, and the reaction container may be able to be joined to an end of the elution container that differs from an end to which the washing container is joined.

According to the container assembly kit, washing, elution, and a reaction can be sequentially effected by joining the reaction container to the elution container. Since the reaction container and the elution container are provided separately, it is possible to reduce the length of the elution container.

### Application Example 13

In the container assembly kit, the washing container may include an insertion section that is situated at one end of a part of the washing container that forms the flow channel, the elution container may include a reception section that is situated at one end of a part of the elution container that forms the flow channel, and part of an outer wall of the insertion section may come in contact with part of an inner wall of the reception section when the washing container and the elution container are joined.

According to the container assembly kit, since part of the outer wall of the insertion section of the washing container comes in contact with part of the inner wall of the reception section of the elution container, it is possible to easily move the substance-binding solid-phase carrier from the washing container to the elution container after assembly.

### Application Example 14

According to another aspect of the invention, a container assembly kit includes:

an adsorption container that seal-tightly holds an adsorbent and a fluid that is immiscible with the adsorbent; and

a washing container that seal-tightly holds a washing liquid and a fluid that is immiscible with the washing liquid, the adsorbent being a liquid in which a nucleic acid is adsorbed on a substance-binding solid-phase carrier, the washing liquid being a liquid with which the substance-binding solid-phase carrier on which a biological substance is adsorbed is washed, and the adsorption container and the washing container being able to be joined to form a flow channel through which the biological substance is moved.

According to the container assembly kit, since the adsorption container and the washing container respectively seal-tightly hold the contents, it is possible to prevent a situation in which the washing liquid is contaminated by the adsorbent until the adsorption container and the washing container are joined.

### Application Example 15

According to another aspect of the invention, a container assembly kit includes:

an adsorption container that seal-tightly holds an adsorbent and a fluid that is immiscible with the adsorbent; and

an elution container that seal-tightly holds an eluent and a fluid that is immiscible with the eluent,

the adsorbent being a liquid in which a nucleic acid is adsorbed on a substance-binding solid-phase carrier,

the eluent being a liquid with which a biological substance is eluted from the substance-binding solid-phase carrier, and

the adsorption container and the elution container being able to be joined to form a flow channel through which the biological substance is moved.

According to the container assembly kit, since the adsorption container and the elution container respectively seal-tightly hold the contents, it is possible to prevent a situation in which the eluent is contaminated by the adsorbent until the adsorption container and the elution container are joined.

### Application Example 16

According to another aspect of the invention, a container assembly kit includes:

a first washing container that seal-tightly holds a first washing liquid and a fluid that is immiscible with the first washing liquid; and

a second washing container that seal-tightly holds a second washing liquid and a fluid that is immiscible with the second washing liquid, the second washing liquid differing from the first washing liquid,

the first washing liquid and the second washing liquid being a liquid with which a substance-binding solid-phase carrier on which a biological substance is adsorbed is washed, and

the first washing container and the second washing container being able to be joined to form a flow channel through which the biological substance is moved.

According to the container assembly kit, since the first washing container and the second washing container respectively seal-tightly hold the contents, it is possible to prevent a situation in which the second washing liquid is contaminated by the first washing liquid until the first washing container and the second washing container are joined.

### BRIEF DESCRIPTION OF DRAWINGS

FIG. 1 is a front view of a container assembly 1 according to one embodiment of the invention.

FIG. 2 is a side view of a container assembly 1 according to one embodiment of the invention.

FIG. 3 is a plan view of a container assembly 1 according to one embodiment of the invention.

FIG. 4 is a perspective view of a container assembly 1 according to one embodiment of the invention.

FIG. 5 is a cross-sectional view of a container assembly 1 according to one embodiment of the invention taken along the line A-A in FIG. 3.

FIG. 6 is a cross-sectional view of a container assembly 1 according to one embodiment of the invention taken along the line C-C in FIG. 3.

FIG. 7A is a schematic view illustrating a method for operating a container assembly 1 according to one embodiment of the invention.

FIG. 7B is a schematic view illustrating a method for operating a container assembly 1 according to one embodiment of the invention.

FIG. 8A is a schematic view illustrating a method for operating a container assembly 1 according to one embodiment of the invention.

FIG. 8B is a schematic view illustrating a method for operating a container assembly 1 according to one embodiment of the invention.

FIG. 9 is a schematic configuration diagram illustrating a PCR device 50.

FIG. 10 is a block diagram illustrating a PCR device 50.

FIG. 11 is a perspective view of a third washing container 230.

FIG. 12 is a vertical cross-sectional view of a third washing container 230.

FIG. 13 is a vertical cross-sectional view of an elution container 300.

FIG. 14 is a vertical cross-sectional view of an adsorption container 100.

FIG. 15 is a vertical cross-sectional view of a reaction container 400.

### DESCRIPTION OF EMBODIMENTS

Several exemplary embodiments of the invention are described below. Note that the following exemplary embodiments merely illustrate examples of the invention. The invention is not limited to the following exemplary embodiments. The invention includes various modifications that can be practiced without departing from the scope of the invention. Note that all of the elements described below in connection with the exemplary embodiments should not necessarily be taken as essential elements of the invention.

A container assembly according to one embodiment of the invention includes a washing container that seal-tightly holds a washing liquid and a fluid that is immiscible with the washing liquid, and an elution container that seal-tightly holds an eluent and a fluid that is immiscible with the eluent, the washing container and the elution container being joined to form a flow channel through which a biological substance is moved, the washing liquid being a liquid with which a substance-binding solid-phase carrier on which the biological substance is adsorbed is washed, and the eluent being a liquid with which the biological substance is eluted from the substance-binding solid-phase carrier.

A container assembly kit according to one embodiment of the invention includes a washing container that seal-tightly holds a washing liquid and a fluid that is immiscible with the washing liquid, and an elution container that seal-tightly holds an eluent and a fluid that is immiscible with the eluent, the washing liquid being a liquid with which a substance-binding solid-phase carrier on which a biological substance is adsorbed is washed, the eluent being a liquid with which the biological substance is eluted from the substance-binding solid-phase carrier, the washing container and the elution container being able to be joined so as to form a flow channel through which the biological substance is moved.

Examples of the biological substance include a biopolymer such as a nucleic acid (DNA and RNA), a polypeptide, a protein, and a polysaccharide, a biological low-molecular-weight organic compound such as a protein, an enzyme, a peptide, a nucleotide, an amino acid, and a vitamin, an inorganic compound, and the like. The embodiments of the invention will be described taking an example in which the biological substance is a nucleic acid.

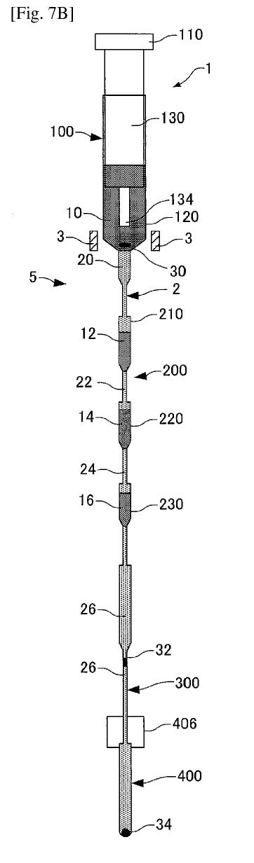

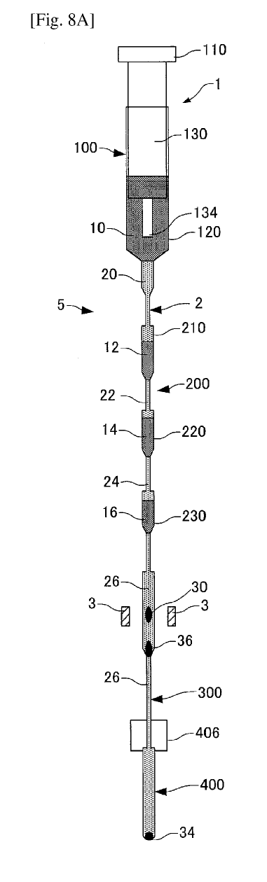

The term “substance-binding solid-phase carrier” used herein refers to a substance that can hold the biological substance through adsorption (i.e., reversible physical binding). It is preferable that the substance-binding solid-phase carrier be microparticles. Note that the substance-binding solid-phase carrier is not limited thereto. For example, the substance-binding solid-phase carrier may be microfibers or a net-like carrier. It is preferable that the substance-binding solid-phase carrier have magnetic properties so that the substance-binding solid-phase carrier can be moved in the desired direction within the container assembly in a state in which the biological substance is adsorbed on the substance-binding solid-phase carrier. The embodiments of the invention will be described taking an example in which the substance-binding solid-phase carrier is a magnetic bead 30 (see FIGS. 7A, 7B, 8A, and 8B) on which a nucleic acid is adsorbed.

The washing liquid 12, 14, 16 (see FIGS. 7A, 7B, 8A, and 8B) is a liquid for washing the substance-binding solid-phase carrier on which the biological substance is adsorbed. It is possible to remove impurities and the like while ensuring that the biological substance is adsorbed on the substance-binding solid-phase carrier in a stable manner by washing the substance-binding solid-phase carrier with the washing liquid.

The fluid that is immiscible with the washing liquid is a fluid that is immiscible with the washing liquid within the washing container, and undergoes phase separation with respect to the washing liquid. The fluid that is immiscible with the washing liquid is a substance that is inert to the washing liquid, and may be a gas such as air. When the washing liquid is an aqueous liquid, an oil, an oil gel, or the like that is immiscible with the aqueous liquid may be used as the fluid that is immiscible with the washing liquid. The term “oil gel” used herein refers to a gel that is obtained by subjecting a liquid oil to gelation using a gellant. Note that the term “oil” used herein excludes an oil gel. The embodiments of the invention will be described taking an example in which the fluid that is immiscible with the washing liquid is an oil 20, 22, 24, 26 (see FIGS. 7A, 7B, 8A, and 8B).

The eluent 32 (see FIGS. 7A, 7B, 8A, and 8B) is a substance with which the biological substance is desorbed and eluted from the substance-binding solid-phase carrier. For example, water or a buffer may be used as the eluent.

The fluid that is immiscible with the eluent is a fluid that is immiscible with the eluent within the elution container, and undergoes phase separation with respect to the eluent. The fluid that is immiscible with the eluent is a substance that is inert to the eluent. The embodiments of the invention will be described taking an example in which the fluid that is immiscible with the eluent is an oil 26 (see FIGS. 7A, 7B, 8A, and 8B).

1. Outline of Container Assembly

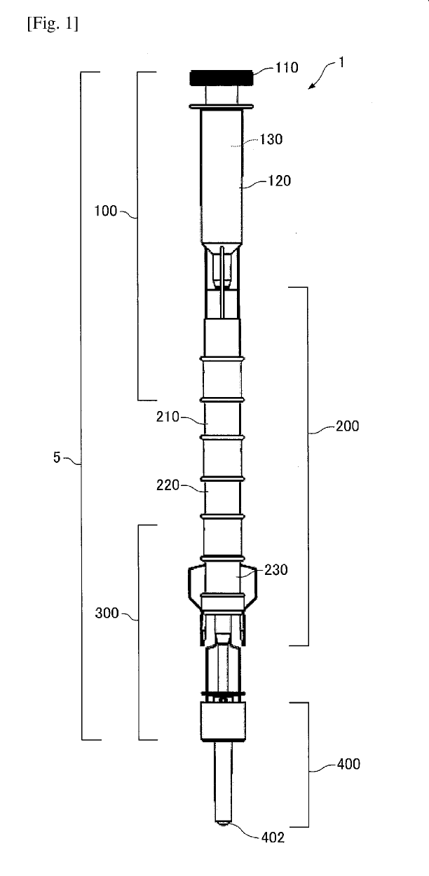

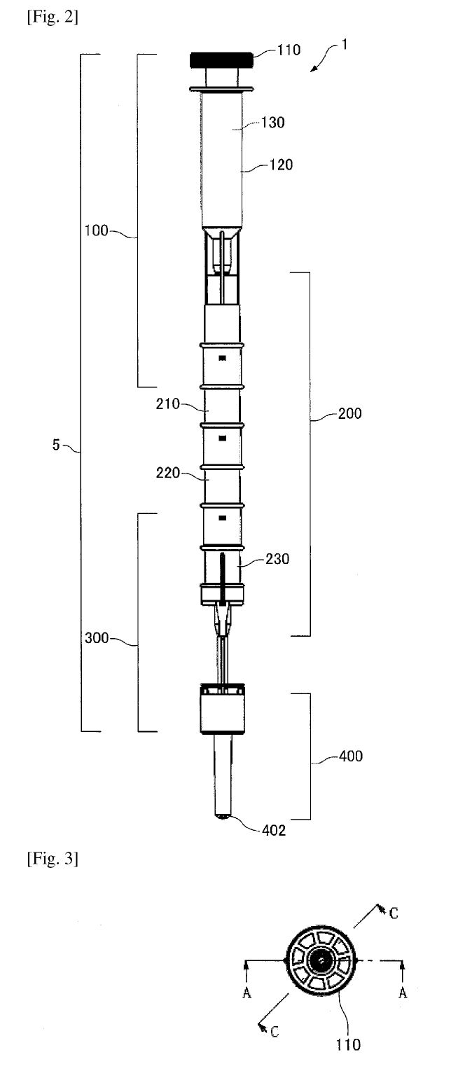

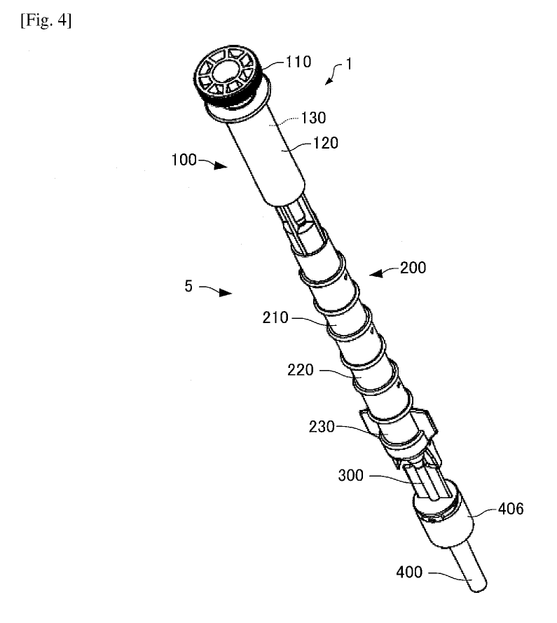

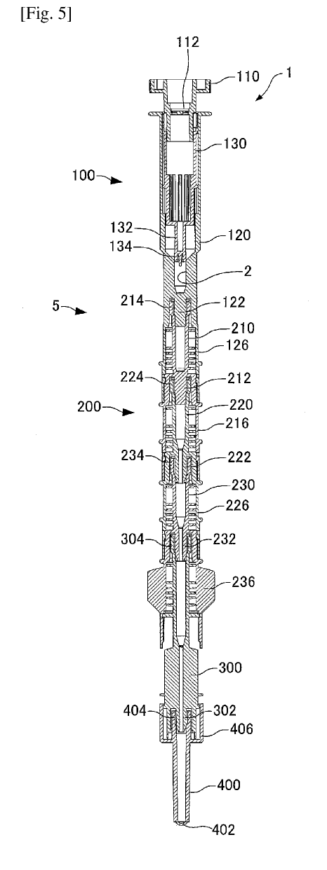

An outline of a container assembly 1 according to one embodiment of the invention is described below with reference to FIGS. 1 to 4. FIG. 1 is a front view illustrating the container assembly 1 (hereinafter may be referred to as “cartridge”) according to one embodiment of the invention. FIG. 2 is a side view illustrating the container assembly 1 according to one embodiment of the invention. FIG. 3 is a plan view illustrating the container assembly 1 according to one embodiment of the invention. FIG. 4 is a perspective view illustrating the container assembly 1 according to one embodiment of the invention. Note that the state of the container assembly 1 illustrated in FIGS. 1 to 3 is referred to as “upright state”.

The container assembly 1 includes an adsorption container 100, a washing container 200, an elution container 300, and a reaction container 400. The container assembly 1 is a container that forms a flow channel (not illustrated in the drawings) that extends (communicates) from the adsorption container 100 to the reaction container 400. The flow channel formed by the container assembly 1 is closed by a cap 110 at one end, and is closed by a bottom 402 at the other end.

The container assembly 1 is designed to effect a pretreatment that causes a nucleic acid to be bound to a magnetic bead (not illustrated in the drawings) within the adsorption container 100, purified while the magnetic bead moves within the washing container 200, and eluted into an eluent droplet (not illustrated in the drawings) within the elution container 300, and subjects the eluent droplet that includes the nucleic acid to PCR thermal cycling within the reaction container 400.

A material for forming the container assembly 1 is not particularly limited. For example, the container assembly 1 may be formed of glass, a polymer, a metal, or the like. It is preferable to form the container assembly 1 using a material (e.g., glass or polymer) that allows visible light to pass through since the inside (cavity) of the container assembly 1 can be observed from the outside. It is preferable to form the container assembly 1 using a material that allows a magnetic force to pass through or a non-magnetic material since the magnetic bead (not illustrated in the drawings) can be easily passed through the container assembly 1 by applying a magnetic force from the outside of the container assembly 1, for example. The container assembly 1 may be formed of a polypropylene resin, for example.

The adsorption container 100 includes a cylindrical syringe section 120 that holds an adsorbent (not illustrated in the drawings), a plunger section 130 that is a movable plunger that is inserted into the syringe section 120, and the cap 110 that is secured on one end of the plunger section 130. The adsorption container 100 is designed so that the plunger section 130 can be slid along the inner surface of the syringe section 120, and the adsorbent (not illustrated in the drawings) contained in the syringe section 120 can be discharged into the washing container 200 by moving the cap 110 toward the syringe section 120. The details of the adsorbent are described later.

The washing container 200 is assembled by joining a first washing container 210, a second washing container 220, and a third washing container 230. Each of the first washing container 210, the second washing container 220, and the third washing container 230 includes one or more washing liquid layers that are partitioned by an oil layer (not illustrated in the drawings). The washing container 200 (assembled by joining the first washing container 210, the second washing container 220, and the third washing container 230) includes a plurality of washing liquid layers that are partitioned by a plurality of oil layers (not illustrated in the drawings). Although an example in which the washing container 200 utilizes the first washing container 210, the second washing container 220, and the third washing container 230 has been described above, the number of washing containers may be appropriately increased or decreased corresponding to the number of washing liquid layers. The details of the washing liquid are described later.

The elution container 300 is joined to the third washing container 230 included in the washing container 200, and holds the eluent so that the shape of a plug can be maintained. The term “plug” used herein refers to a specific liquid when the specific liquid occupies a space (compartment) within a flow channel. More specifically, the plug of a specific liquid refers to a pillar-shaped space that is substantially occupied by only the specific liquid (i.e., the space within the flow channel is partitioned by the plug of the liquid). The expression “substantially” used in connection with the plug means that a small amount (e.g., thin film) of another substance (e.g., liquid) may be present around the plug (i.e., on the inner wall of the flow channel). The details of the eluent are described later.

A nucleic acid purification device 5 includes the adsorption container 100, the washing container 200, and the elution container 300.

The reaction container 400 is joined to the elution container 300, and receives a liquid discharged from the elution container 300. The reaction container 400 holds the eluent droplet that includes a sample during thermal cycling. The reaction container 400 also holds a reagent (not illustrated in the drawings). The details of the reagent are described later.

2. Details of Structure of Container Assembly

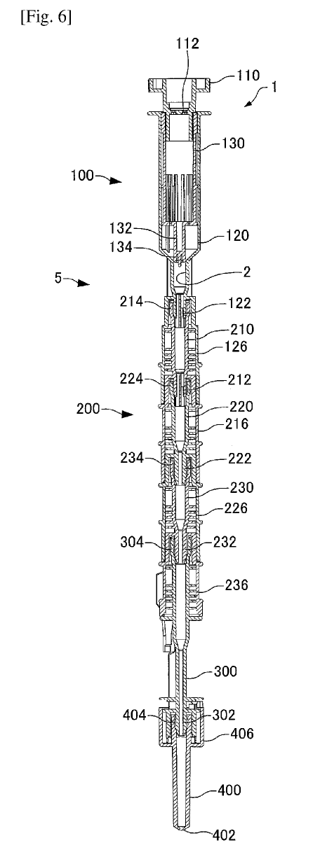

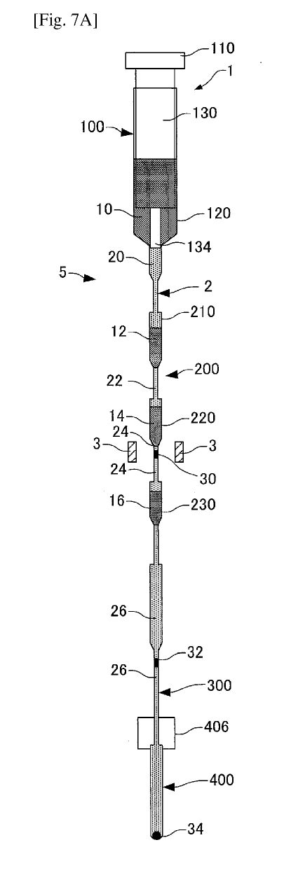

The details of the structure of the container assembly 1 are described below with reference to FIGS. 5 and 6. FIG. 5 is a cross-sectional view of the container assembly 1 according to one embodiment of the invention taken along the line A-A in FIG. 3. FIG. 6 is a cross-sectional view of the container assembly 1 according to one embodiment of the invention taken along the line C-C in FIG. 3. Note that the container assembly 1 is assembled in a state in which each container is charged with the washing liquid or the like. In FIGS. 5 and 6, the washing liquid and the like are omitted so that the structure of the container assembly 1 can be easily understood.

2-1. Adsorption Container

The adsorption container 100 has a structure in which the plunger section 130 is inserted into the syringe section 120 through one open end of the syringe section 120, and the cap 110 is inserted into the open end of the plunger section 130. The cap 110 has a vent section 112 that is provided at the center thereof. The vent section 112 suppresses a change in the internal pressure of the plunger section 130 when the plunger section 130 is operated.

The plunger section 130 is an approximately cylindrical plunger that slides along the inner circumferential surface of the syringe section 120. The plunger section 130 includes the open end into which the cap 110 is inserted, a rod-like section 132 that extends from the bottom situated opposite to the open end in the longitudinal direction of the syringe section 120, and an end section 134 that is provided at the end of the rod-like section 132. The rod-like section 132 protrudes from the center of the bottom of the plunger section 130. A through-hole is formed in the wall of the rod-like section 132 so that the inner space of the plunger section 130 communicates with the inner space of the syringe section 120.

The syringe section 120 forms part of a flow channel 2 of the container assembly 1. The syringe section 120 includes a large-diameter section that holds the plunger section 130, a small-diameter section that is smaller in inner diameter than the large-diameter section, a diameter reduction section that is provided between the large-diameter section and the small-diameter section and decreases in inner diameter, an adsorption insertion section 122 that is provided at the end of the small-diameter section, and a cylindrical adsorption cover section 126 that covers the adsorption insertion section 122. The large-diameter section, the small-diameter section, and the adsorption insertion section 122 that form part of the flow channel 2 of the container assembly 1 have an approximately cylindrical shape.

The end section 134 of the plunger section 130 seals the small-diameter section of the syringe section 120 (when the container assembly 1 is provided to the worker) to divide the large-diameter section and the diameter reduction section from the small-diameter section (i.e., divide the syringe section 120 into two compartments).

The adsorption insertion section 122 of the syringe section 120 is inserted and fitted into a first reception section 214 that forms one open end of the first washing container 210 included in the washing container 200 to join the syringe section 120 and the first washing container 210. The outer circumferential surface of the adsorption insertion section 122 comes in close contact with the inner circumferential surface of the first reception section 214 to prevent leakage of a liquid to the outside.

2-2. Washing Container

The washing container 200 forms part of the flow channel 2 of the container assembly 1, and includes the first washing container 210, the second washing container 220, and the third washing container 230 (i.e., is assembled by joining the first washing container 210, the second washing container 220, and the third washing container 230). The first washing container 210, the second washing container 220, and the third washing container 230 have an identical basic structure. Therefore, only the structure of the first washing container 210 is described below, and description of the structure of the second washing container 220 and the structure of the third washing container 230 is omitted.

The first washing container 210 has an approximately cylindrical shape, and extends in the longitudinal direction of the container assembly 1. The first washing container 210 includes a first insertion section 212 that is formed at one open end, the first reception section 214 that is formed at the other open end, and a cylindrical first cover section 216 that covers the first insertion section 212.

The outer diameter of the first insertion section 212 is approximately the same as the inner diameter of a second reception section 224. The inner diameter of the first reception section 214 is approximately the same as the outer diameter of the adsorption insertion section 122.

When the first insertion section 212 of the first washing container 210 is inserted and fitted into the second reception section 224 of the second washing container 220, the outer circumferential surface of the first insertion section 212 comes in close contact with (i.e., seals) the inner circumferential surface of the second reception section 224, and the first washing container 210 is joined to the second washing container 220. The first washing container 210, the second washing container 220, and the third washing container 230 are thus joined (connected) to form the washing container 200. The term “seal” used herein refers to sealing a container or the like so that at least a liquid or gas contained in the container or the like does not leak to the outside. The term “seal” used herein may include sealing a container or the like so that a liquid or gas does not enter the container or the like from the outside.

2-3. Elution Container

The elution container 300 has an approximately cylindrical shape, and extends in the longitudinal direction of the container assembly 1. The elution container 300 forms part of the flow channel 2 of the container assembly 1. The elution container 300 includes an elution insertion section 302 that is formed at one open end, and an elution reception section 304 that is formed at the other open end.

The inner diameter of the elution reception section 304 is approximately the same as the outer diameter of a third insertion section 232 of the third washing container 230. When the third insertion section 232 is inserted and fitted into the elution reception section 304, the outer circumferential surface of the third insertion section 232 comes in close contact with (i.e., seals) the inner circumferential surface of the elution reception section 304, and the third washing container 230 is joined to the elution container 300.

2-4. Reaction Container

The reaction container 400 has an approximately cylindrical shape, and extends in the longitudinal direction of the container assembly 1. The reaction container 400 forms part of the flow channel 2 of the container assembly 1. The reaction container 400 includes a reaction reception section 404 that is formed at the open end, a bottom 402 that is formed at the closed end (that is situated opposite to the open end), and a reservoir section 406 that covers the reaction reception section 404.

The inner diameter of the reaction reception section 404 is approximately the same as the outer diameter of the elution insertion section 302 of the elution container 300. When the elution insertion section 302 is inserted and fitted into the reaction reception section 404, the elution container 300 is joined to the reaction container 400.

The reservoir section 406 has a predetermined space, and is provided around the reaction reception section 404. The reservoir section 406 has a capacity sufficient to receive a liquid that overflows the reaction container 400 due to the movement of the plunger section 130.

3. Contents of Container Assembly, and Method for Operating Container Assembly

The contents of the container assembly 1 are described below with reference to FIG. 7A, and a method for operating the container assembly 1 is described below with reference to FIGS. 7A, 7B, 8A, and 8B. FIGS. 7A and 7B are schematic views illustrating the method for operating the container assembly 1 according to one embodiment of the invention. FIGS. 8A and 8B are schematic views illustrating the method for operating the container assembly 1 according to one embodiment of the invention. In FIGS. 7A, 7B, 8A, and 8B, each container is represented by the flow channel 2, and the external shape and the joint (junction) structure of each container are omitted so that the state of the contents can be easily understood.

3-1. Contents

FIG. 7A illustrates the state of the contents of the flow channel 2 when the container assembly 1 is set to the state illustrated in FIG. 1. An adsorbent 10, a first oil 20, a first washing liquid 12, a second oil 22, a second washing liquid 14, a third oil 24, a magnetic bead 30, the third oil 24, a third washing liquid 16, a fourth oil 26, an eluent 32, the fourth oil 26, and a reagent 34 are included in the flow channel 2 sequentially from the cap 110 to the reaction container 400.

The flow channel 2 has a structure in which parts (i.e., thick parts) having a large cross-sectional area (in a plane that is orthogonal to the longitudinal direction of the container assembly 1) and parts (i.e., thin parts) having a small cross-sectional area (in a plane that is orthogonal to the longitudinal direction of the container assembly 1) are provided alternately. The thin parts of the flow channel 2 respectively hold part or the entirety of the first oil 20, the second oil 22, the third oil 24, the fourth oil 26, and the eluent 32. The thin parts of the flow channel 2 have a cross-sectional area that ensures that the interface between liquids (may be fluids (hereinafter the same)) that are contiguous to each other and are immiscible with each other can be maintained within the thin part in a stable manner. Therefore, the relationship between a liquid situated within the thin part of the flow channel 2 and another liquid that is contiguous thereto can be maintained in a stable manner due to the liquid situated within the thin part. Even when the interface between a liquid situated within the thin part of the flow channel 2 and another liquid situated within the thick part of the flow channel 2 is formed within the thick part of the flow channel 2, the interface is formed at a predetermined position in a stable manner even if the interface is affected by a high impact by allowing the liquids to stand.

The thin part of the flow channel 2 is formed within the adsorption insertion section 122, the first insertion section 212, the second insertion section 222, the third insertion section 232, and the elution insertion section 302. In the elution container 300, the thin part of the flow channel 2 extends upward beyond the elution insertion section 302. Note that a liquid held within the thin part of the flow channel 2 is maintained in a stable manner even prior to assembly.

3-1-1. Oil

The first oil 20, the second oil 22, the third oil 24, and the fourth oil 26 include an oil, and are present in the form of a plug between the liquids contiguous thereto in the state illustrated in FIGS. 7A and 7B. A liquid that undergoes phase separation with respect to each oil (i.e., a liquid that is immiscible with each oil) is selected as the liquid contiguous to each oil so that the first oil 20, the second oil 22, the third oil 24, and the fourth oil 26 are present in the form of a plug. The first oil 20, the second oil 22, the third oil 24, and the fourth oil 26 may differ in the type of oil. An oil selected from a silicone-based oil (e.g., dimethyl silicone oil), a paraffinic oil, a mineral oil, and a mixture thereof may be used as the first oil 20, the second oil 22, the third oil 24, and the fourth oil 26, for example.

3-1-2. Adsorbent

The adsorbent 10 is a liquid in which the nucleic acid is adsorbed on the magnetic bead 30. For example, the adsorbent 10 is an aqueous solution that includes a chaotropic substance (material). 5 M guanidine thiocyanate, 2% Triton X-100, or 50 mM Tris-HCl (pH: 7.2) may be used as the adsorbent 10, for example. The adsorbent 10 is not particularly limited as long as the adsorbent 10 includes a chaotropic substance. A surfactant may be added to the adsorbent 10 in order to destroy a cell membrane, or denature proteins included in a cell. The surfactant is not particularly limited as long as the surfactant is normally used for extraction of a nucleic acid from a cell or the like. Specific examples of the surfactant include a nonionic surfactant such as a Triton-based surfactant (e.g., Triton-X) and a Tween-based surfactant (e.g., Tween 20), and an anionic surfactant such as sodium N-lauroyl sarcosinate (SDS). It is preferable to use a nonionic surfactant at a concentration of 0.1 to 2%. It is preferable that the adsorbent 10 include a reducing agent such as 2-mercaptoethanol or dithiothreitol. The solvent may be a buffer. It is preferable that the solvent have a pH of 6 to 8 (i.e., neutral region). It is preferable that the adsorbent 10 include a guanidine salt (3 to 7 M), a nonionic surfactant (0 to 5%), EDTA (0 to 0.2 mM), a reducing agent (0 to 0.2 M), and the like taking the above points into consideration.

The chaotropic substance is not particularly limited as long as the chaotropic substance produces chaotropic ions (i.e., monovalent anions having a large ionic radius) in an aqueous solution to increase the water solubility of hydrophobic molecules, and contributes to adsorption of the nucleic acid on the solid-phase carrier. Specific examples of the chaotropic substance include guanidine hydrochloride, sodium iodide, sodium perchlorate, and the like. It is preferable to use guanidine thiocyanate or guanidine hydrochloride that exhibits a high protein denaturation effect. These chaotropic substances are used at a different concentration. For example, guanidine thiocyanate is preferably used at a concentration of 3 to 5.5 M, and guanidine hydrochloride is preferably used at a concentration of 5 M or more.

When the chaotropic substance is present in the aqueous solution, the nucleic acid included in the aqueous solution is adsorbed on the surface of the magnetic bead 30 since it is thermodynamically advantageous for the nucleic acid to be adsorbed on a solid rather than being enclosed by water molecules.

3-1-3. Washing Liquid

The first washing liquid 12, the second washing liquid 14, and the third washing liquid 16 are used to wash the magnetic bead 30 on which the nucleic acid is adsorbed.

The first washing liquid 12 is a liquid that undergoes phase separation with respect to the first oil 20 and the second oil 22. It is preferable that the first washing liquid 12 be water or an aqueous solution having a low salt concentration. When using an aqueous solution having a low salt concentration as the first washing liquid 12, a buffer is preferably used as the first washing liquid 12. The salt concentration in the aqueous solution having a low salt concentration is preferably 100 mM or less, more preferably 50 mM or less, and most preferably 10 mM or less. The first washing liquid 12 may include a surfactant (see above). The pH of the first washing liquid 12 is not particularly limited. The salt that may be used for the first washing liquid 12 (buffer) is not particularly limited. It is preferable to use Tris, HEPES, PIPES, phosphoric acid, or the like. It is preferable that the first washing liquid 12 include an alcohol in such an amount that adsorption of the nucleic acid on the carrier, a reverse transcription reaction, PCR, and the like are not hindered. In this case, the alcohol concentration in the first washing liquid 12 is not particularly limited.

The first washing liquid 12 may include a chaotropic substance. For example, when the first washing liquid 12 includes guanidine hydrochloride, the magnetic bead 30 or the like can be washed while maintaining or strengthening adsorption of the nucleic acid on the magnetic bead 30 or the like.

The second washing liquid 14 is a liquid that undergoes phase separation with respect to the second oil 22 and the third oil 24. The second washing liquid 14 may have the same composition as that of the first washing liquid 12, or may have a composition differing from that of the first washing liquid 12. It is preferable that the second washing liquid 14 be a solution that substantially does not include a chaotropic substance. This is because it is preferable to prevent a situation in which a chaotropic substance is incorporated in the subsequent solution. For example, a 5 mM Tris-HCl buffer may be used as the second washing liquid 14. It is preferable that the second washing liquid 14 include an alcohol (see above).

The third washing liquid 16 is a liquid that undergoes phase separation with respect to the third oil 24 and the fourth oil 26. The third washing liquid 16 may have the same composition as that of the second washing liquid 14, or may have a composition differing from that of the second washing liquid 14. Note that the third washing liquid 16 does not include an alcohol. The third washing liquid 16 may include citric acid in order to prevent a situation in which an alcohol enters the reaction container 400.

3-1-4. Magnetic Bead

The magnetic bead 30 is a bead on which the nucleic acid is adsorbed. It is preferable that the magnetic bead 30 have relatively high magnetic properties so that the magnetic bead 30 can be moved using a magnet 3 that is provided outside the container assembly 1. The magnetic bead 30 may be a silica bead or a silica-coated bead, for example. The magnetic bead 30 may preferably be a silica-coated bead.

3-1-5. Eluent

The eluent 32 is a liquid that undergoes phase separation with respect to the fourth oil 26. The eluent 32 is present in the form of a plug that is situated between the fourth oil 26 within the flow channel 2 included in the elution container 300. The eluent 32 is a liquid with which the nucleic acid adsorbed on the magnetic bead 30 is eluted from the magnetic bead 30. The eluent 32 forms a droplet within the fourth oil 26 due to heating. For example, purified water may be used as the eluent 32. Note that the term “droplet” used herein refers to a liquid that is enclosed by a free surface.

3-1-6. Reagent

The reagent 34 includes a component necessary for a reaction. When effecting PCR within the reaction container 400, the reagent 34 may include at least one of an enzyme (e.g., DNA polymerase) and a primer (nucleic acid) for amplifying the target nucleic acid (DNA) eluted into the eluent droplet 36 (see FIGS. 8A and 8B), and a fluorescent probe for detecting the amplified product. For example, the reagent 34 includes all of the primer, the enzyme, and the fluorescent probe. The reagent 34 is incompatible with the fourth oil 26. The reagent 34 is dissolved upon contact with the droplet 36 of the eluent 32 including the nucleic acid, and undergoes a reaction. The reagent 34 is present in a solid state in the lowermost part of the flow channel 2 (within the reaction container 400) in the gravitational direction. For example, a freeze-dried reagent may be used as the reagent 34.

3-2. Method for Operating Container Assembly

An example of the method for operating the container assembly 1 is described below with reference to FIGS. 7A, 7B, 8A, and 8B.

The method for operating the container assembly 1 includes (A) joining the adsorption container 100, the washing container 200, the elution container 300, and the reaction container 400 to assemble the container assembly 1 (hereinafter may be referred to as “step (A)”), (B) introducing a sample that includes the nucleic acid into the adsorption container 100 that holds the adsorbent 10 (hereinafter may be referred to as “step (B)”), (C) moving the magnetic bead 30 from the second washing container 220 to the adsorption container 100 (hereinafter may be referred to as “step (C)”), (D) causing the nucleic acid to be adsorbed on the magnetic bead 30 by shaking the adsorption container 100 (hereinafter may be referred to as “step (D)”), (E) moving the magnetic bead 30 on which the nucleic acid is adsorbed from the adsorption container 100 to the elution container 300 sequentially through the first oil 20, the first washing liquid 12, the second oil 22, the second washing liquid 14, the third oil 24, the third washing liquid 16, and the fourth oil 26 (hereinafter may be referred to as “step (E)”), (F) eluting the nucleic acid adsorbed on the magnetic bead 30 into the eluent 32 within the elution container 300 (hereinafter may be referred to as “step (F)”), and (G) bringing the droplet that includes the nucleic acid into contact with the reagent 34 included in the reaction container 400 (hereinafter may be referred to as “step (G)”).

Each step is described below.

Step (A) that Assembles Container Assembly 1

In the step (A), the adsorption container 100, the washing container 200, the elution container 300, and the reaction container 400 are joined to assemble the container assembly 1 so that the flow channel 2 is formed to extend from the adsorption container 100 to the reaction container 400 (see FIG. 7A). Although FIG. 7A illustrates a state in which the cap 110 is fitted to the adsorption container 100, the cap 110 is fitted to the plunger section 130 after the step (B).

More specifically, the elution insertion section 302 of the elution container 300 is inserted into the reaction reception section 404 of the reaction container 400, the third insertion section 232 of the third washing container 230 is inserted into the elution reception section 304 of the elution container 300, the second insertion section 222 of the second washing container 220 is inserted into the third reception section 234 of the third washing container 230, the first insertion section 212 of the first washing container 210 is inserted into the second reception section 224 of the second washing container 220, and the adsorption insertion section 122 of the adsorption container 100 is inserted into the first reception section 214 of the first washing container 210.

Step (B) that Introduces Sample

In the step (B), a cotton swab that holds the sample is put into the adsorbent 10 through the opening of the adsorption container 100 into which the cap 110 is fitted, and immersed in the adsorbent 10, for example. More specifically, the cotton swab is inserted into the adsorption container 100 through the opening formed at one end of the plunger section 130 that is inserted into the syringe section 120. After removing the cotton swab from the adsorption container 100, the cap 110 is fitted into the adsorption container 100 (see FIG. 7A). The sample may be introduced into the adsorption container 100 using a pipette or the like. When the sample is in the form of a paste or a solid, the sample may be put into the adsorption container 100 (or caused to adhere to the inner wall of the plunger section 130) using a spoon, tweezers, or the like. As illustrated in FIG. 7A, the syringe section 120 and the plunger section 130 are not completely filled with the adsorbent 10, and an empty space is formed on the side of the opening into which the cap 110 is fitted.

The sample includes the nucleic acid that is the target (hereinafter may be referred to as “target nucleic acid”). The target nucleic acid is either or both of deoxyribonucleic acid (DNA) and ribonucleic acid (RNA), for example. The target nucleic acid is extracted from the sample, eluted into the eluent 32 (described later), and used as a PCR template, for example. Examples of the sample include a biological sample such as blood, nasal mucus, and an oral mucous membrane, and the like.

Step (C) that Moves Magnetic Bead

In the step (C), the magnetic bead 30 that is situated between the third oil 24 and present in the form of a plug within the second washing container 220 is moved by moving the magnet 3 (that is disposed outside the container) toward the adsorption container 100 in a state in which a magnetic force is applied using the magnet 3 (see FIG. 7A).

The cap 110 and the plunger section 130 are moved in the direction away from the syringe section 120 when moving the magnetic bead 30 (or before moving the magnetic bead 30) to move the sample included in the adsorbent 10 from the plunger section 130 to the syringe section 120. The flow channel 2 that has been closed by the end section 134 communicates with the adsorbent 10 as a result of moving the plunger section 130.

The magnetic bead 30 moves upward within the flow channel 2 along with the movement of the magnet 3, and reaches the adsorbent 10 that includes the sample (see FIG. 7B).

Step (D) that Causes Nucleic Acid to be Adsorbed on Magnetic Bead

In the step (D), the adsorption container 100 is shaken. The step (D) can be efficiently performed since the opening of the adsorption container 100 is sealed with the cap 110 so that the adsorbent 10 does not leak. The target nucleic acid is thus adsorbed on the surface of the magnetic bead 30 due to the effect of the chaotropic agent. In the step (D), a nucleic acid other than the target nucleic acid and proteins may be adsorbed on the surface of the magnetic bead 30.

The adsorption container 100 may be shaken using a known vortex shaker or the like, or may be shaken manually. The adsorption container 100 may be shaken while applying a magnetic field from the outside by utilizing the magnetic properties of the magnetic bead 30.

Step (E) that Moves Magnetic Bead on which Nucleic Acid is Adsorbed

In the step (E), the magnetic bead 30 is moved through the adsorbent 10, the first oil 20, the second oil 22, the third oil 24, the fourth oil 26, the first washing liquid 12, the second washing liquid 14, and the third washing liquid 16 while applying a magnetic force generated by the magnet 3 from the outside of the adsorption container 100, the washing container 200, and the elution container 300.

For example, a permanent magnet, an electromagnet, or the like may be used as the magnet 3. The magnet 3 may be moved manually, or may be moved using a mechanical device or the like. The magnetic bead 30 is moved within the flow channel 2 through the adsorption container 100, the washing container 200, and the elution container 300 while changing the relative position of the magnet 3 by utilizing the fact that the magnetic bead 30 is attracted by a magnetic force. The speed at which the magnetic bead 30 is passed through each washing liquid is not particularly limited. The magnetic bead 30 may be moved forward and backward within an identical washing liquid along the longitudinal direction of the flow channel 2. Note that a particle or the like other than the magnetic bead 30 may be moved within the tube by utilizing gravity or a potential difference, for example.

Step (F) that Elutes Nucleic Acid

In the step (F), the nucleic acid is eluted from the magnetic bead 30 into the eluent droplet 36 within the elution container 300. In FIGS. 7A and 7B, the eluent 32 is present in the form of a plug within the thin part of the flow channel included in the elution container 300. The eluent droplet 36 moves upward within the elution container 300 (see FIGS. 8A and 8B) since the contents of the reaction container 400 expand as a result of heating the reaction container 400 while moving the magnetic bead 30. When the magnetic bead 30 has reached the eluent droplet 36 included in the elution container 300, the target nucleic acid adsorbed on the magnetic bead 30 is eluted into the eluent droplet 36 due to the effect of the eluent (see FIG. 8A).

Step (G) that Brings Droplet that Includes Nucleic Acid into Contact with Reagent 34

In the step (G), the droplet 36 that includes the nucleic acid is brought into contact with the reagent 34 that is situated in the lowermost part of the reaction container 400. Specifically, the first oil 20 is pushed downward using the end section 134 of the plunger section 130 by moving the cap 110 downward. The eluent droplet 36 into which the target nucleic acid has been eluted thus enters the reaction container 400, and comes in contact with the reagent 34 that is situated in the lowermost part of the reaction container 400 in a state in which the magnetic bead 30 to which a magnetic force generated by the magnet 3 is applied is maintained at a predetermined position (see FIG. 8B). The reagent 34 that has come in contact with the droplet 36 is dissolved, and mixed with the target nucleic acid included in the eluent. PCR that utilizes thermal cycling is thus effected, for example.

4. PCR Device

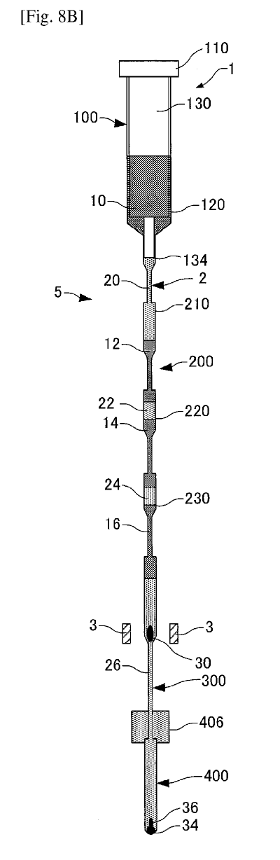

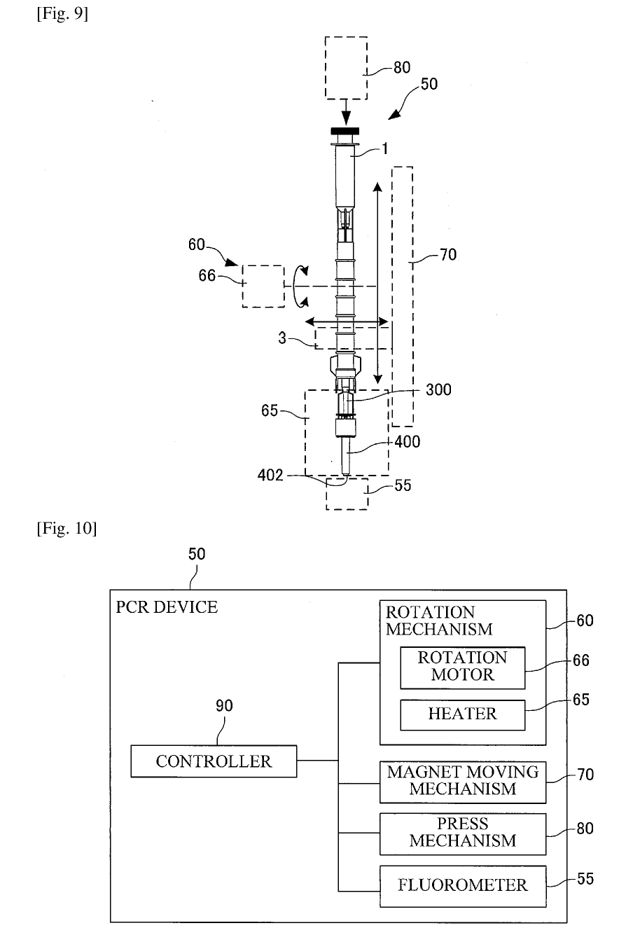

A PCR device 50 that implements a nucleic acid elution process and PCR using the container assembly 1 is described below with reference to FIGS. 9 and 10. FIG. 9 is a schematic configuration diagram illustrating the PCR device 50. FIG. 10 is a block diagram illustrating the PCR device 50.

The PCR device 50 includes a rotation mechanism 60, a magnet moving mechanism 70, a press mechanism 80, a fluorometer 55, and a controller 90.

4-1. Rotation Mechanism

The rotation mechanism 60 includes a rotation motor 66 and a heater 65, and rotates the container assembly 1 and the heater 65 by driving the rotation motor 66. When the container assembly 1 and the heater 65 are rotated (flipped upside down) by the rotation mechanism 60, the droplet that includes the target nucleic acid moves within the flow channel included in the reaction container 400, and subjected to thermal cycling.

The heater 65 includes a plurality of heaters (not illustrated in the drawings). For example, the heater 65 may include an elution heater, a high-temperature heater, and a low-temperature heater. The elution heater heats the eluent (that is present in the form of a plug) included in the container assembly 1 to promote elution of the target nucleic acid from the magnetic bead into the eluent. The high-temperature heater heats the upstream-side liquid within the flow channel included in the reaction container 400 to a temperature higher than that achieved by the low-temperature heater. The low-temperature heater heats the bottom 402 of the reaction container 400 (flow channel). It is possible to provide the liquid within the flow channel included in the reaction container 400 with a temperature gradient by utilizing the high-temperature heater and the low-temperature heater. The heater 65 is provided with a temperature controller, and can set the liquid within the container assembly 1 to a temperature suitable for the process according to an instruction from the controller 90.

The heater 65 has an opening that exposes the outer wall of the bottom 402 of the reaction container 400. The fluorometer 55 measures the brightness of the eluent droplet through the opening.

4-2. Magnet Moving Mechanism

The magnet moving mechanism 70 moves the magnet 3. The magnet moving mechanism 70 moves the magnetic bead within the container assembly 1 by moving the magnet 3 in a state in which the magnet 3 attracts the magnetic bead within the container assembly 1. The magnet moving mechanism 70 includes a pair of magnets 3, an elevating mechanism, and a swing mechanism.

The swing mechanism swings the pair of magnets 3 in the transverse direction (or the forward-backward direction) in FIG. 9. The pair of magnets 3 are disposed on either side of the container assembly 1 fitted to the PCR device 50 (see FIGS. 7A, 7B, 8A, and 8B). The distance between the magnetic bead and each magnet 3 can be reduced in the direction (transverse direction in FIG. 9) orthogonal to the flow channel of the container assembly 1. When the pair of magnets 3 are swung in the transverse direction (see the two-headed arrow), the magnetic bead within the container assembly 1 moves in the transverse direction along with the movement of the pair of magnets 3. The elevating mechanism moves the magnetic bead in the vertical direction in FIG. 9 by moving the magnet 3 in the vertical direction.

4-3. Press Mechanism

The press mechanism 80 presses the plunger section included in the container assembly 1. When the plunger section is pressed by the press mechanism 80, the droplet within the elution container 300 is discharged into the reaction container 400, and PCR is effected within the reaction container 400.

In FIG. 9, the press mechanism 80 is disposed above the container assembly 1 that is set to an upright state. Note that the press mechanism 80 may press the plunger section in the direction that is tilted by 45o with respect to the vertical direction, for example. This makes it possible to easily dispose the press mechanism 80 at a position at which the press mechanism 80 does not interfere with the magnet moving mechanism 70.

4-4. Fluorometer

The fluorometer 55 measures the brightness of the droplet within the reaction container 400. The fluorometer 55 is disposed at a position opposite to the bottom 402 of the reaction container 400. It is desirable that the fluorometer 55 be able to detect the brightness within a plurality of wavelength bands so that multiplex PCR can be implemented.

4-5. Controller

The controller 90 is a control section that controls the PCR device 50. The controller 90 includes a processor (e.g., CPU) and a storage device (e.g., ROM and RAM). Various programs and data are stored in the storage device. The storage device provides an area into which a program is loaded. Various processes are implemented by causing the processor to execute the program stored in the storage device.

For example, the controller 90 rotates the container assembly 1 to a predetermined rotation position by controlling the rotation motor 66. A rotation position sensor (not illustrated in the drawings) is provided to the rotation mechanism 60. The controller 90 drives and stops the rotation motor 66 corresponding to the detection results of the rotation position sensor.

The controller 90 heats the liquid within the container assembly 1 to a predetermined temperature by ON/OFF-controlling the heater 65.

The controller 90 moves the magnet 3 in the vertical direction by controlling the magnet moving mechanism 70, and swings the magnet 3 in the transverse direction in FIG. 9 corresponding to the detection results of a position sensor (not illustrated in the drawings).

The controller 90 measures the brightness of the droplet within the reaction container 400 by controlling the fluorometer 55. The measurement results are stored in a storage device (not illustrated in the drawings) included in the controller 90.

The container assembly 1 is fitted to the PCR device 50, and the steps (C) to (G) (see “3-2. Method for operating container assembly”) and PCR are effected.

5. Container Assembly Kit

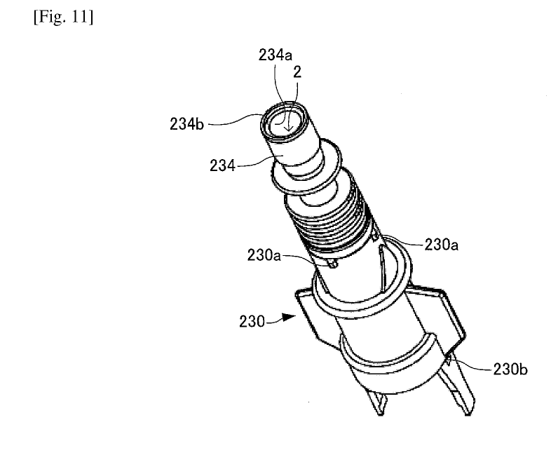

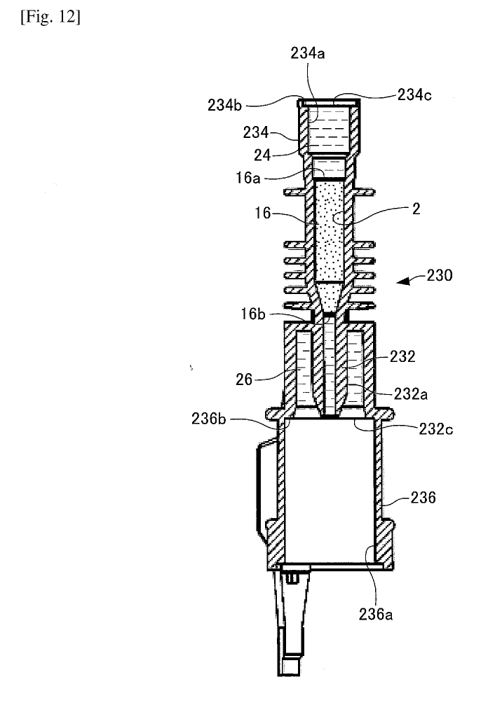

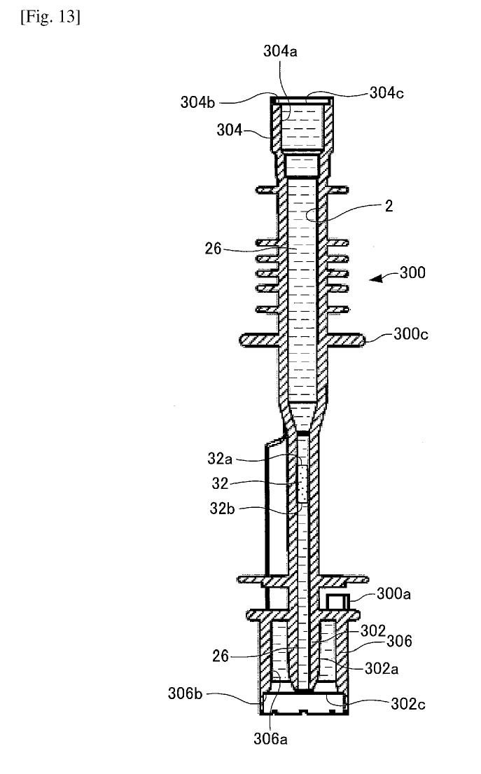

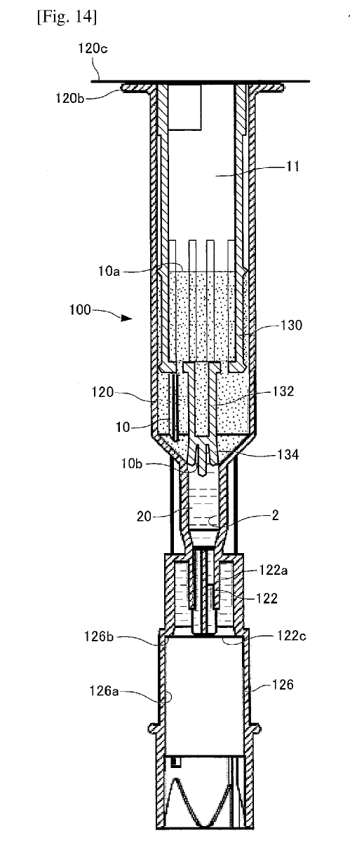

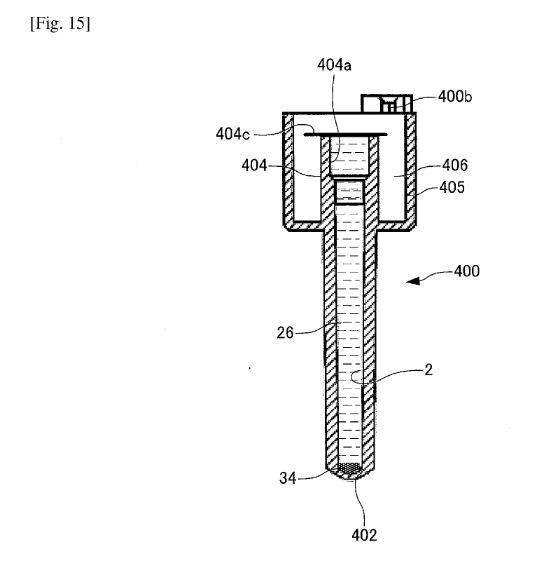

The container assembly kit according to one embodiment of the invention is described below with reference to FIGS. 11 to 15. FIG. 11 is a perspective view of the third washing container 230. FIG. 12 is a vertical cross-sectional view of the third washing container 230. FIG. 13 is a vertical cross-sectional view of the elution container 300. FIG. 14 is a vertical cross-sectional view of the adsorption container 100. FIG. 15 is a vertical cross-sectional view of the reaction container 400. The container assembly kit illustrated in FIGS. 11 to 15 is used to assemble the container assembly 1 illustrated in FIGS. 1 to 8B. Note that description of the same features as those described above in connection with the container assembly 1 is omitted.

The container assembly kit includes the third washing container 230 illustrated in FIGS. 11 and 12 and the elution container 300 illustrated in FIG. 13. The container assembly kit is characterized in that the third washing container 230 and the elution container 300 can be joined so as to form the flow channel 2 through which the target nucleic acid is moved. Note that the third washing container 230 is the minimum constituent unit of the washing container.

5-1. Washing Container

As illustrated in FIGS. 11 and 12, the third washing container 230 (i.e., washing container) seal-tightly holds the third washing liquid 16 (i.e., washing liquid) as well as the third oil 24 and the fourth oil 26 (i.e., a fluid that is immiscible with the third washing liquid 16).

The third washing container 230 includes the third insertion section 232 (that is situated at one end of the part of the third washing container 230 that forms the flow channel 2) and the third reception section 234 (that is situated at the other end of the part of the third washing container 230 that forms the flow channel 2). The flow channel 2 is formed through the third washing container 230 from the third insertion section 232 to the third reception section 234. The outer diameter of the flow channel 2 gradually decreases from the third reception section 234 toward the third insertion section 232.

The third insertion section 232 has an approximately cylindrical shape, and has an outer wall 232a having a circular horizontal cross-sectional shape.

The third washing container 230 includes a third cover section 236 that is formed around the third insertion section 232, and opens downward from the upper part of the outer wall 232a.

The upper end of the third cover section 236 is connected to the outer wall 232a of the third insertion section 232, and the lower end of the third cover section 236 extends beyond the third insertion section 232. An inner wall 236a of the third cover section 236 has a circular step 236b at which the diameter of the inner wall 236a increases. The step 236b is situated at a position slightly lower than the lower end of the third insertion section 232, and a film 232c is bonded to the surface of the step 236b.

The third reception section 234 has an approximately cylindrical shape, and has an inner wall 234a having a circular horizontal cross-sectional shape. The inner wall 234a has a tubular step 234b at which the diameter of the inner wall 234a increases. The step 234b is situated in the vicinity of the upper end of the third reception section 234, and a film 234c is bonded to the surface of the step 234b. Note that the film 234c is omitted in FIG. 11.

The upper opening and the lower opening of the third washing container 230 are respectively sealed with the film 234c and the film 232c in a state in which the third oil 24, the third washing liquid 16, and the fourth oil 26 are held within the flow channel 2 sequentially from the third reception section 234. The third washing liquid 16 and the third oil 24 are not mixed with each other at an interface 16a, and the third washing liquid 16 and the fourth oil 26 are not mixed with each other at an interface 16b. Therefore, the third oil 24, the third washing liquid 16, and the fourth oil 26 are seal-tightly held within the third washing container 230 so that the third washing liquid 16 is held in the form of a plug.

5-2. Elution Container

As illustrated in FIG. 13, the elution container 300 seal-tightly holds the eluent 32 and the fourth oil 26 (i.e., a fluid that is immiscible with the eluent 32).

The shape of the elution container 300 is basically the same as that of the third washing container 230.

The elution container 300 includes the elution insertion section 302 (that is situated at one end of the part of the elution container 300 that forms the flow channel 2) and the elution reception section 304 (that is situated at the other end of the part of the elution container 300 that forms the flow channel 2). The flow channel 2 is formed through the elution container 300 from the elution insertion section 302 to the elution reception section 304. The outer diameter of the flow channel 2 gradually decreases from the elution reception section 304 toward the elution insertion section 302.

The elution insertion section 302 has an approximately cylindrical shape, and has an outer wall 302a having a circular horizontal cross-sectional shape.

The elution container 300 includes an elution cover section 306 that is formed around the elution insertion section 302, and opens downward from the upper part of the outer wall 302a.

The upper end of the elution cover section 306 is connected to the outer wall 302a of the elution insertion section 302, and the lower end of the elution cover section 306 extends beyond the elution insertion section 302. An inner wall 306a of the elution cover section 306 has a circular step 306b at which the diameter of the inner wall 306a increases. The step 306b is situated at a position slightly lower than the lower end of the elution insertion section 302, and a film 302c is bonded to the surface of the step 306b.

The elution reception section 304 has an approximately cylindrical shape, and has an inner wall 304a having a circular horizontal cross-sectional shape. The inner wall 304a has a tubular step 304b at which the diameter of the inner wall 304a increases. The step 304b is situated in the vicinity of the upper end of the elution reception section 304, and a film 304c is bonded to the surface of the step 304b.

The upper opening and the lower opening of the elution container 300 are respectively sealed with the film 304c and the film 302c in a state in which the fourth oil 26, the eluent 32, and the fourth oil 26 are held within the flow channel 2 sequentially from the elution reception section 304. The eluent 32 and the fourth oil 26 situated on the upper side of the eluent 32 are not mixed with each other at an interface 32a, and the eluent 32 and the fourth oil 26 situated on the lower side of the eluent 32 are not mixed with each other at an interface 32b. Therefore, the fourth oil 26 and the eluent 32 are seal-tightly held within the elution container 300 so that the eluent 32 is held in the form of a plug.

According to the container assembly kit, since the third washing container 230 and the elution container 300 respectively seal-tightly hold the contents, it is possible to prevent a situation in which the eluent 32 is contaminated by the third washing liquid 16 until the third washing container 230 and the elution container 300 are joined.

The container assembly 1 illustrated in FIGS. 1 to 8B that has been assembled using the container assembly kit includes the third washing container 230 (or the washing container 200) and the elution container 300. The third washing container 230 and the elution container 300 are joined to form the flow channel 2 through which the target nucleic acid is moved. According to the container assembly 1, since the third washing container 230 and the elution container 300 respectively seal-tightly hold the contents until the third washing container 230 and the elution container 300 are joined, it is possible to prevent a situation in which the eluent 32 is contaminated by the third washing liquid 16. According to the container assembly 1, since the third washing liquid 16 and the eluent 32 are not mixed with each other due to the fourth oil 26 (that is immiscible with the third washing liquid 16 and the eluent 32) even after the third washing container 230 and the elution container 300 have been joined, it is possible to prevent a situation in which the eluent 32 is contaminated by the third washing liquid 16 on condition that the container assembly 1 is used immediately after assembly.

5-3. Joint Structure

Part of the outer wall of one end of the part of the third washing container 230 that forms the flow channel 2 is brought into contact with part of the inner wall of one end of the part of the elution container 300 that forms the flow channel 2. More specifically, the third washing container 230 and the elution container 300 are joined in a state in which part of the outer wall 232a of the third insertion section 232 of the third washing container 230 comes in contact with part of the inner wall 304a of the elution reception section 304. The container assembly 1 may be sealed so that the contents of the container assembly 1 do not leak from the flow channel 2 to the outside in an area in which part of the outer wall 232a comes in contact with part of the inner wall 304a.

When the third washing container 230 and the elution container 300 are joined, the third insertion section 232 is inserted into the elution reception section 304 while the third insertion section 232 and the elution reception section 304 break the film 232c and the film 304c. Specifically, the flow channel 2 included in the third washing container 230 and the flow channel 2 included in the elution container 300 do not communicate with each other until the third insertion section 232 and the elution reception section 304 break the film 232c and the film 304c.

Since the container assembly kit is configured so that the outer wall 232a of the third insertion section 232 of the third washing container 230 comes in contact with the inner wall 304a of the elution reception section 304 of the elution container 300, it is possible to easily move the target nucleic acid from the third washing container 230 to the elution container 300 after assembly. Since it is difficult to join two containers that have an end having an identical size, a step is formed within the flow channel 2. In particular, since the target nucleic acid is moved using the magnetic bead 30 having a small size, the movement of the magnetic bead 30 is significantly limited if the diameter of the flow channel 2 decreases in the moving direction at the step. Since the container assembly kit is configured so that the outer wall 232a comes in contact with the inner wall 304a, the diameter of the flow channel 2 does not decrease in the direction from the third washing container 230 toward the elution container 300, and the magnetic bead 30 can be easily moved.

The outer wall 232a and the inner wall 304a may include a truncated conical shape in which the outer diameter slightly decreases in the downward direction. It is possible to achieve a more reliable seal due to the contact between the outer wall 232a and the inner wall 304a.

5-4. Separate Washing Container

As illustrated in FIGS. 1 to 8B (container assembly 1), the washing container 200 included in the container assembly kit may include the first washing container 210, the second washing container 220, and the third washing container 230 (i.e., two or more separate washing containers), the first washing container 210 seal-tightly holding the first washing liquid 12 (i.e., washing liquid) and the second oil 22 (i.e., a fluid that is immiscible with the first washing liquid 12), the second washing container 220 seal-tightly holding the second washing liquid 14 (i.e., washing liquid) as well as the second oil 22 and the third oil 24 (i.e., a fluid that is immiscible with the second washing liquid 14), and the third washing container 230 seal-tightly holding the third washing liquid 16 (i.e., washing liquid) as well as the third oil 24 and the fourth oil 26 (i.e., a fluid that is immiscible with the third washing liquid 16).

The first washing container 210 and the second washing container 220 are basically configured in the same manner as the third washing container 230 (see above).

The container assembly kit may be configured so that the second washing container 220 (i.e., separate washing container) can be joined to the third washing container 230 (i.e., separate washing container) that is joined to the elution container 300 (see FIGS. 5 and 6). The container assembly kit may be configured so that the first washing container 210 can be joined to the second washing container 220.

This makes it possible to prevent a situation in which each washing liquid is contaminated until the first washing container 210, the second washing container 220, and the third washing container 230 (i.e., separate washing containers) are joined. When the container assembly kit includes the first washing container 210, the second washing container 220, and the third washing container 230 (i.e., two or more separate washing containers), it is possible to adjust the washing accuracy corresponding to the user's demand.

According to the container assembly 1 that is assembled using the container assembly kit, since the first washing liquid 12, the second washing liquid 14, and the third washing liquid 16 are not mixed with each other due to the presence of the first oil 20, the second oil 22, the third oil 24, and the fourth oil 26 even after the first washing container 210, the second washing container 220, and the third washing container 230 have been joined, it is possible to prevent a situation in which the first washing liquid 12, the second washing liquid 14, and the third washing liquid 16 are contaminated on condition that the container assembly 1 is used immediately after assembly. According to the container assembly 1, since the magnetic bead 30 can be washed two or more times using the first washing liquid 12, the second washing liquid 14, and the third washing liquid 16 that are respectively held by the first washing container 210, the second washing container 220, and the third washing container 230, it is possible to improve the washing efficiency.

5-5. Adsorption Container

The container assembly kit may further include the adsorption container 100 that seal-tightly holds the adsorbent 10 and the first oil 20 (i.e., a fluid that is immiscible with the adsorbent 10) (see FIG. 14).

The adsorption container 100 can be joined to the third reception section 234. The third washing container 230 includes the third insertion section 232 (i.e., one end to which the elution container 300 is joined) and the third reception section 234 (i.e., the other end that differs from the third insertion section 232). When three separate washing containers are used as the washing container 200 (see the container assembly 1 illustrated in FIGS. 1 to 8B), the other end is the first reception section 214 of the first washing container 210.

The adsorption container 100 has a structure in which the plunger section 130 is inserted into the syringe section 120, and a film 120c is bonded to the upper side of a flange 120b that is situated at the upper end of the syringe section 120. The syringe section 120 includes the adsorption insertion section 122 that is situated at one end, and the flange 120b that is situated at the other end and has a circular shape that extends outward. The adsorption insertion section 122 has an approximately cylindrical shape, and has an outer wall 122a having a circular horizontal cross-sectional shape.

The syringe section 120 includes an adsorption cover section 126 that is formed around the adsorption insertion section 122, and opens downward from the upper part of the outer wall 122a. The upper end of the adsorption cover section 126 is connected to the outer wall 122a of the adsorption insertion section 122, and the lower end of the adsorption cover section 126 extends beyond the adsorption insertion section 122. An inner wall 126a of the adsorption cover section 126 has a circular step 126b at which the diameter of the inner wall 126a increases. The step 126b is situated at a position slightly lower than the lower end of the adsorption insertion section 122, and a film 122c is bonded to the surface of the step 126b.

The upper opening and the lower opening of the adsorption container 100 are respectively sealed with the film 120c and the film 122c in a state in which air 11, the adsorbent 10, and the first oil 20 are held within the flow channel 2 sequentially from the flange 120b. The adsorbent 10 and the first oil 20 are not mixed with each other at an interface 10b. Since the flow channel 2 is sealed with the end section 134, the adsorbent 10 does not move. When the adsorption container 100 is stationary, the adsorbent 10 and the air 11 are not mixed with each other at an interface 10a (free interface).

When the adsorption container 100 and the third washing container 230 are joined, the adsorption insertion section 122 is inserted into the third reception section 234 while the adsorption insertion section 122 and the third reception section 234 break the film 122c and the film 234c. Specifically, the flow channel 2 included in the adsorption container 100 and the flow channel 2 included in the third washing container 230 do not communicate with each other until the adsorption insertion section 122 and the third reception section 234 break the film 122c and the film 234c.

According to the container assembly kit, since the adsorption container 100 seal-tightly holds the contents until the adsorption container 100 and the third washing container 230 are joined, it is possible to prevent a situation in which the third washing liquid 16 is contaminated by the adsorbent 10.

According to the container assembly 1 (see FIGS. 1 to 8B) that is assembled using the container assembly kit, since the adsorbent 10, the first washing liquid 12, the second washing liquid 14, and the third washing liquid 16 are not mixed with each other due to the presence of the first oil 20, the second oil 22, the third oil 24, and the fourth oil 26 even after the adsorption container 100 and the washing container 200 have been joined, it is possible to prevent a situation in which the first washing liquid 12, the second washing liquid 14, and the third washing liquid 16 are contaminated by the adsorbent 10 on condition that the container assembly 1 is used immediately after assembly.

5-6. Reaction Container

The container assembly kit may further include the reaction container 400 that seal-tightly holds the fourth oil 26 that is immiscible with the eluent 32 (see FIG. 15).

The container assembly kit may be configured so that the reaction container 400 can be joined to the elution insertion section 302 (i.e., the other end that differs from the elution reception section 304 (i.e., one end) to which the third washing container 230 is joined) of the elution container 300.

The reaction container 400 includes the bottom 402 (that is situated at one end) and the reaction reception section 404 (that is situated at the other end). The reaction reception section 404 has an approximately cylindrical shape, and has an inner wall 404a having a circular horizontal cross-sectional shape. The reaction container 400 includes a reaction cover section 405 that is formed around the reaction reception section 404, and opens upward from the lower part of the reaction reception section 404. The lower end of the reaction cover section 405 is connected to the outer wall of the reaction reception section 404, and the upper end of the reaction cover section 405 extends beyond the reaction reception section 404. A film 404c is bonded to the upper side of the reaction reception section 404.

The opening of the reaction container 400 sealed with the film 404c in a state in which the fourth oil 26 (situated on the side of the reaction reception section 404) and the reagent 34 (situated on the side of the bottom 402) are held within the flow channel 2.

When the reaction container 400 and the elution container 300 are joined, the elution insertion section 302 is inserted into the reaction reception section 404 while the elution insertion section 302 and the reaction reception section 404 break the film 302c and the film 404c. Specifically, the flow channel 2 included in the elution container 300 and the flow channel 2 included in the reaction container 400 do not communicate with each other until the elution insertion section 302 and the reaction reception section 404 break the film 302c and the film 404c.

According to the container assembly kit, washing, elution, and a reaction (e.g., nucleic acid amplification reaction) can be sequentially effected by joining the reaction container 400 to the elution container 300. Since the reaction container 400 and the elution container 300 are provided separately, it is possible to reduce the length of the elution container 300 as compared with the case where the elution container 300 also serves as a reaction container.

The container assembly 1 illustrated in FIGS. 1 to 8B can be assembled using the container assembly kit (i.e., the container assembly 1 can be obtained by assembling the container assembly kit).

5-7. Fitting Structure

The container joint structure is designed so that the insertion section of each container is inserted into the reception section of the adjacent container (i.e., the insertion section comes in contact with the reception section) to effect a seal (see above). A fitting structure may be provided in addition to the joint structure in order to more reliably maintain the joint structure. A snap-fit structure may be used as the fitting structure.

The snap-fit structure implements mechanical joining by utilizing the elasticity of a material. In this case, each container has an engagement protrusion that protrudes from the outer side of the container, and an engagement hole that receives the engagement protrusion. In FIG. 11, the third washing container 230 has an engagement protrusion 230a and an engagement hole 230b, for example. The engagement protrusion 230a is fitted into an engagement hole (not illustrated in the drawings) formed in the second washing container 220 to hold the third washing container 230 and the second washing container 220. An engagement protrusion (not illustrated in the drawings) provided to an engagement flange 300c (see FIG. 13) of the elution container 300 is fitted into the engagement hole 230b to hold the third washing container 230 and the elution container 300. FIG. 13 illustrates an engagement protrusion 300a that is provided to the elution insertion section 302, and FIG. 15 illustrates an engagement hole 400b into which the engagement protrusion 300a is fitted.

The invention is not limited to the above embodiments. Various modifications and variations may be made of the above embodiments without departing from the scope of the invention. For example, the invention includes various other configurations that are substantially the same as the configurations described in connection with the above embodiments (e.g., a configuration having the same function, method, and results, or a configuration having the same objective and results). The invention also includes a configuration in which an unsubstantial element described in connection with the above embodiments is replaced by another element. The invention also includes a configuration having the same effects as those of the configurations described in connection with the above embodiments, or a configuration capable of achieving the same objective as that of the configurations described in connection with the above embodiments. The invention further includes a configuration in which a known technique is added to the configurations described in connection with the above embodiments.

6. Modifications

Although an example in which the container assembly kit (see “5. Container assembly kit”) includes the third washing container 230 and the elution container 300 as the minimum constituent units, the invention is not limited thereto. Several modifications of the above embodiments are described below. The following modifications illustrate an example in which the combination of the containers included in the container assembly 1 (see “1. Outline of container assembly” to “4. PCR device”) and the container assembly kit (see “5. Container assembly kit”) (see FIGS. 1 to 15) is changed. The details described above are also applied to the following modifications.

6-1. First Modification

A container assembly kit according to a first modification includes the adsorption container 100 that seal-tightly holds the adsorbent 10 and the first oil 20 (i.e., a fluid that is immiscible with the adsorbent 10), and the first washing container 210 (i.e., washing container) that seal-tightly holds the first washing liquid 12 (i.e., washing liquid) and the second oil 22 (i.e., a fluid that is immiscible with the first washing liquid 12), the adsorption container 100 and the first washing container 210 being able to be joined so as to form the flow channel 2 through which the biological substance is moved. The first washing container 210 is basically configured in the same manner as the third washing container 230 (see FIGS. 11 and 12), and detailed description thereof is omitted. According to the container assembly kit, since the adsorption container 100 and the first washing container 210 respectively seal-tightly hold the contents, it is possible to prevent a situation in which the washing liquid 12 is contaminated by the adsorbent 10 until the adsorption container 100 and the first washing container 210 are joined.

The container assembly 1 is assembled using the container assembly kit according to the first modification in the same manner as described above with reference to FIGS. 1 to 8B. When the adsorption container 100 and the first washing container 210 (i.e., minimum constituent units) are assembled (joined), a film is bonded to the first insertion section 212 of the first washing container 210 in the same manner as in the third washing container 230 illustrated in FIG. 12, and the second washing container 220 and the like are omitted. According to such a container assembly 1, the effect achieved by the container assembly kit can also be achieved. Moreover, since the first washing liquid 12 and the adsorbent 10 are not mixed with each other due to the presence of the fluid (first oil 20) that is immiscible with the first washing liquid 12 and the adsorbent 10 even after the adsorption container 100 and the first washing container 210 have been joined, it is possible to prevent a situation in which the first washing liquid 12 is contaminated by the adsorbent 10 on condition that the container assembly 1 is used immediately after assembly.

The container assembly kit and the container assembly 1 according to the first modification may include a plurality of separate washing containers (see “5-4. Separate washing container”). The container assembly kit and the container assembly 1 according to the first modification may further include the elution container 300 (see “5-2. Elution container”) and the reaction container 400 (see “5-6. Reaction container”).

6-2. Second Modification

A container assembly kit according to a second modification includes the adsorption container 100 that seal-tightly holds the adsorbent 10 and the first oil 20 (i.e., a fluid that is immiscible with the adsorbent 10), and the elution container 300 that seal-tightly holds the eluent 32 and the fourth oil 26 (i.e., a fluid that is immiscible with the eluent 32), the adsorption container 100 and the elution container 300 being able to be joined so as to form the flow channel 2 through which the biological substance is moved. According to the container assembly kit, since the adsorption container 100 and the elution container 300 respectively seal-tightly hold the contents, it is possible to prevent a situation in which the eluent 32 is contaminated by the adsorbent 10 until the adsorption container 100 and the elution container 300 are joined.

A container assembly assembled using the container assembly kit according to the second modification has a configuration in which the washing container 200 and the reaction container 400 are omitted from the container assembly 1 illustrated in FIGS. 1 to 8B, and the adsorption container 100 is joined directly to the elution container 300. Specifically, the adsorption insertion section 122 of the adsorption container 100 is inserted into the elution reception section 304 of the elution container 300. According to the resulting container assembly, the target nucleic acid can be adsorbed on the magnetic bead 30, the magnetic bead 30 can be moved to the elution container 300, and the target nucleic acid can be eluted from the magnetic bead 30 into the eluent 32 without performing the washing step as long as impurities can be removed from the target nucleic acid to a predetermined level within the adsorbent 10 (or before the target nucleic acid is introduced into the adsorbent 10). According to the container assembly, the effect achieved by the container assembly kit can also be achieved. Moreover, since the eluent 32 and the adsorbent 10 are not mixed with each other due to the presence of the fluid (first oil 20 and fourth oil 26) that is immiscible with the eluent 32 and the adsorbent 10 even after the adsorption container 100 and the elution container 300 have been joined, it is possible to prevent a situation in which the eluent 32 is contaminated by the adsorbent 10 on condition that the container assembly 1 is used immediately after assembly.

The container assembly kit and the container assembly according to the second modification may further include the reaction container 400 (see “5-6. Reaction container”).

6-3. Third Modification

A container assembly kit according to a third modification includes the first washing container 210 that seal-tightly holds the first washing liquid 12 and the first oil 20 and/or the second oil 22 (i.e., a fluid that is immiscible with the first washing liquid 12), and the second washing container 220 that seal-tightly holds the second washing liquid 14 (that differs from the first washing liquid 12) and the second oil 22 and/or the third oil 24 (i.e., a fluid that is immiscible with the second washing liquid 14), the first washing container 210 and the second washing container 220 being able to be joined so as to form the flow channel 2 through which the biological substance is moved. The first washing container 210 and the second washing container 220 are basically configured in the same manner as the third washing container 230 (see FIGS. 11 and 12), and detailed description thereof is omitted. According to the container assembly kit, since the first washing container 210 and the second washing container 220 respectively seal-tightly hold the contents, it is possible to prevent a situation in which the second washing liquid 14 is contaminated by the first washing liquid 12 until the first washing container 210 and the second washing container 220 are joined.

The container assembly 1 is assembled using the container assembly kit according to the third modification in the same manner as described above with reference to FIGS. 1 to 8B. When the first washing container 210 and the second washing container 220 (i.e., minimum constituent units) are assembled (joined), a film is bonded to the first reception section 214 of the first washing container 210 and the second insertion section 222 of the second washing container 220 in the same manner as in the third washing container 230 illustrated in FIG. 12, and the adsorption container 100, the third washing container 230, and the like are omitted. According to such a container assembly 1, the effect achieved by the container assembly kit can also be achieved. Moreover, since the first washing container 210 and the second washing container 220 respectively seal-tightly hold the contents, it is possible to prevent a situation in which the second washing liquid 14 is contaminated by the first washing liquid 12 until the first washing container 210 and the second washing container 220 are joined.

The container assembly kit and the container assembly according to the third modification may have a configuration in which the third washing container 230 (see “5-1. Washing container”) is joined to the second insertion section 222. The container assembly kit and the container assembly according to the third modification may further include at least one of the elution container 300 (see “5-2. Elution container”), the adsorption container 100 (see “5-5. Adsorption container”), and the reaction container 400 (see “5-6. Reaction container”).

1. A container assembly comprising:
a flow channel through which a biological substance is moved, the flow channel being formed by joining a washing container that seal-tightly holds a washing liquid and a fluid that is immiscible with the washing liquid, and an elution container that seal-tightly holds an eluent and a fluid that is immiscible with the eluent,
the washing liquid being a liquid with which a substance-binding solid-phase carrier on which the biological substance is adsorbed is washed, and
the eluent being a liquid with which the biological substance is eluted from the substance-binding solid-phase carrier.

2. The container assembly as defined in claim 1,
the washing container including two or more separate washing containers that respectively seal-tightly hold the washing liquid and the fluid that is immiscible with the washing liquid,
a separate washing container among the two or more separate washing containers being joined to the elution container, and another separate washing container among the two or more separate washing containers being joined to the separate washing container that is joined to the elution container.

3. The container assembly as defined in claim 1, further comprising:
an adsorption container that seal-tightly holds an adsorbent and a fluid that is immiscible with the adsorbent, the adsorption container being joined to the washing container to form the flow channel,
the adsorbent being a liquid in which a nucleic acid is adsorbed on the substance-binding solid-phase carrier, and
the adsorption container being joined to an end of the washing container that differs from an end to which the elution container is joined.

4. The container assembly as defined in claim 1, further comprising:
a reaction container that seal-tightly holds a fluid that is immiscible with the eluent, the reaction container being joined to the elution container to form the flow channel,
the reaction container being joined to an end of the elution container that differs from an end to which the washing container is joined.

5. The container assembly as defined in claim 1,
one end of a part of the washing container that forms the flow channel being inserted into one end of a part of the elution container that forms the flow channel, and
part of an outer wall of the one end of the part of the washing container that forms the flow channel coming in contact with part of an inner wall of the one end of the part of the elution container that forms the flow channel.

6. A container assembly comprising:
a flow channel through which a biological substance is moved, the flow channel being formed by joining an adsorption container that seal-tightly holds an adsorbent and a fluid that is immiscible with the adsorbent, and a washing container that seal-tightly holds a washing liquid and a fluid that is immiscible with the washing liquid,
the adsorbent being a liquid in which a nucleic acid is adsorbed on a substance-binding solid-phase carrier, and
the washing liquid being a liquid with which the substance-binding solid-phase carrier on which the biological substance is adsorbed is washed.

7. A container assembly comprising:
a flow channel through which a biological substance is moved, the flow channel being formed by joining an adsorption container that seal-tightly holds an adsorbent and a fluid that is immiscible with the adsorbent, and an elution container that seal-tightly holds an eluent and a fluid that is immiscible with the eluent,
the adsorbent being a liquid in which a nucleic acid is adsorbed on a substance-binding solid-phase carrier, and
the eluent being a liquid with which the biological substance is eluted from the substance-binding solid-phase carrier.

8. A container assembly comprising:
a flow channel through which a biological substance is moved, the flow channel being formed by joining a first washing container that seal-tightly holds a first washing liquid and a fluid that is immiscible with the first washing liquid, and a second washing container that seal-tightly holds a second washing liquid and a fluid that is immiscible with the second washing liquid, the second washing liquid differing from the first washing liquid,
the first washing liquid and the second washing liquid being a liquid with which a substance-binding solid-phase carrier on which the biological substance is adsorbed is washed.

9. A container assembly kit comprising:
a washing container that seal-tightly holds a washing liquid and a fluid that is immiscible with the washing liquid; and
an elution container that seal-tightly holds an eluent and a fluid that is immiscible with the eluent,
the washing liquid being a liquid with which a substance-binding solid-phase carrier on which the biological substance is adsorbed is washed,
the eluent being a liquid with which the biological substance is eluted from the substance-binding solid-phase carrier, and
the washing container and the elution container being able to be joined to form a flow channel through which the biological substance is moved.

10. The container assembly kit as defined in claim 9,
the washing container including two or more separate washing containers that respectively seal-tightly hold the washing liquid and the fluid that is immiscible with the washing liquid,
a separate washing container among the two or more separate washing containers being able to be joined to the elution container, and another separate washing container among the two or more separate washing containers being able to be joined to the separate washing container that is joined to the elution container.

11. The container assembly kit as defined in claim 9, further comprising:
an adsorption container that seal-tightly holds an adsorbent and a fluid that is immiscible with the adsorbent,
the adsorbent being a liquid in which a nucleic acid is adsorbed on the substance-binding solid-phase carrier, and
the adsorption container being able to be joined to an end of the washing container that differs from an end to which the elution container is joined.

12. The container assembly kit as defined in claim 9, further comprising:
a reaction container that seal-tightly holds a fluid that is immiscible with the eluent,
the reaction container being able to be joined to an end of the elution container that differs from an end to which the washing container is joined.

13. The container assembly kit as defined in claim 9,
the washing container including an insertion section that is situated at one end of a part of the washing container that forms the flow channel,
the elution container including a reception section that is situated at one end of a part of the elution container that forms the flow channel, and
part of an outer wall of the insertion section coming in contact with part of an inner wall of the reception section when the washing container and the elution container are joined.

14. A container assembly kit comprising:
an adsorption container that seal-tightly holds an adsorbent and a fluid that is immiscible with the adsorbent; and
a washing container that seal-tightly holds a washing liquid and a fluid that is immiscible with the washing liquid,
the adsorbent being a liquid in which a nucleic acid is adsorbed on a substance-binding solid-phase carrier,
the washing liquid being a liquid with which the substance-binding solid-phase carrier on which a biological substance is adsorbed is washed, and
the adsorption container and the washing container being able to be joined to form a flow channel through which the biological substance is moved.

15. A container assembly kit comprising:
an adsorption container that seal-tightly holds an adsorbent and a fluid that is immiscible with the adsorbent; and
an elution container that seal-tightly holds an eluent and a fluid that is immiscible with the eluent,
the adsorbent being a liquid in which a nucleic acid is adsorbed on a substance-binding solid-phase carrier,
the eluent being a liquid with which a biological substance is eluted from the substance-binding solid-phase carrier, and
the adsorption container and the elution container being able to be joined to form a flow channel through which the biological substance is moved.

16. A container assembly kit comprising:
a first washing container that seal-tightly holds a first washing liquid and a fluid that is immiscible with the first washing liquid; and
a second washing container that seal-tightly holds a second washing liquid and a fluid that is immiscible with the second washing liquid, the second washing liquid differing from the first washing liquid,
the first washing liquid and the second washing liquid being a liquid with which a substance-binding solid-phase carrier on which a biological substance is adsorbed is washed, and
the first washing container and the second washing container being able to be joined to form a flow channel through which the biological substance is moved.
